(19) United States
(12) Patent Application Publication
(10) Pub. No.: US 2025/0213398 A1
(43) Pub. Date: Jul. 3, 2025

(54) FIXATION SYSTEM FOR DISPOSABLE ARTICLES

(71) Applicant: Avery Dennison Corporation, Mentor, OH (US)

(72) Inventors: Anne VERHAERT, Turnhout (BE); Johan VAN STEEN, Ravels (BE); Bert BOGAERTS, Boechout (BE); Loes MICHIELSEN, Hoogstraten (BE); Marco LABONIA, Antwerp (BE); Paul DHONDT, Turnhout (BE)

(21) Appl. No.: 18/853,480

(22) PCT Filed: Apr. 27, 2023
(86) PCT No.: PCT/IB2023/054392

§ 371 Date: Oct. 2, 2024

Related U.S. Application Data
(60) Provisional application No. 63/363,775, filed on Apr. 28, 2022.

(51) Int. Cl. A61F 13/505; A61F 13/56; A61F 13/58; A61F 13/62

(52) U.S. Cl. CPC A61F 13/505; A61F 13/5622; A61F 13/58; A61F 13/625

(57) ABSTRACT

Fixation systems that may be found in connection with disposable absorbent articles such as hybrid diapers, as well as other applications. Also disclosed are articles including the fixation systems, and methods of using the fixation systems.

### CROSS-REFERENCE TO RELATED APPLICATION(S)

The present application claims the benefit of U.S. Provisional Patent Application No. 63/363,775, filed Apr. 28, 2022, which is incorporated herein by reference in its entirety.

### TECHNICAL FIELD

The present disclosure relates generally to fixation systems that may be found in connection with disposable absorbent articles such as hybrid diapers, as well as other applications, specifically fastening assemblies. Also disclosed are articles including the fixation systems, and methods of using the fixation systems.

### BACKGROUND

Conventionally, infant diapers have been one of two types, either washable cloth diapers or disposable diapers made of synthetic materials. Currently, the vast majority of infant diapers purchased are of the disposable type. Existing disposable diapers have a liquid impervious outer layer, an inner non-woven liner, and an integral layer of absorbent material sandwiched between the inner and outer layers. Standard disposable diapers are neither re-usable nor recyclable. If the diaper is wetted or soiled, the entire diaper is discarded. Thus, a single user will utilize several diapers during the course of a single day. Accordingly, the single-use nature of the disposable diapers is associated with the creation of a large volume of waste, as well as considerable purchasing costs.

Meanwhile, cloth diapers are environmentally friendly, as they are washable and reusable. Cloth diapers are intended to have a long useable life. In the case of children, the useable life is typically the period of time from the birth of an infant to when the child is trained and no longer requires diapers. Thus, few to no replacements are required for each child. However, cloth diapers come with several disadvantages. Machine washable cloth diapers require significant time and labor to wash them. Additionally, the initial expense of purchasing good quality fitted washable diapers is quite high, compared to the initial cost of disposable diapers.

To address these issues, a variety of diaper systems have been developed that combine some features of disposable diapers with those of cloth, reusable diapers. These systems provide a diaper that is partly reusable and partly disposable. Some of the conventional hybrid diaper systems include two pieces, namely, a re-usable non-absorbent shell, and a separate disposable absorbent pad in the central crotch area, which connects to the shell by an attachment system such as an adhesive tape or buttons. Other hybrid diaper systems have three pieces, including a re-usable non-absorbent shell, a pouch that attaches to the outer shell, and a disposable absorbent pad that can be inserted into the pouch. The pouch and the disposable absorbent pad typically have approximately the same length and width, and the pouch typically has an elasticized border. Thus, the pad is retained within the pouch, without a need for a mechanism to attach the disposable pad to the rest of the diaper system.

Current systems for attaching the disposable absorbent pad to the diaper shell are associated with a variety of disadvantages. Conventional systems utilizing adhesive attachment systems require the use of a liner on the adhesive. This adds a complexity to the handling of the diaper system, and requires the disposal of the liner, which is not environmentally friendly. Further, conventional systems leave adhesive residue on the outer shell when the disposable pad is detached from the diaper shell.

There remains a need for alternative mechanisms to securely attach the disposable pad to the other components of hybrid diaper systems, while addressing the issues associated with conventional hybrid diaper systems. In particular, there remains a need for fastening assemblies to securely attach the disposable pad to the other components of hybrid diaper systems.

### SUMMARY

The embodiments of the present disclosure described below are not intended to be exhaustive or to limit the invention to the precise forms disclosed in the following description. Rather, the embodiments are chosen and described so that others skilled in the art may appreciate and understand the principles and practices of the present disclosure.

Improved fixation devices are shown and described. The fixation device includes at least one bonding component including at least one fixed portion including a first face and an oppositely directed second face; at least one fold-back portion comprising a distal region; and optionally, at least one fastening component connected to the distal region of the bonding component fold-back portion; wherein disposed on at least a portion of the second face of the fixed portion is an adhesive, wherein the distal region of the fold-back portion includes a first face and an oppositely directed second face, wherein the fastening component includes a first face and an oppositely directed second face, wherein disposed on at least one of, at least a portion of the distal region of the fold-back portion, and at least a portion of the fastening component, is at least one fastening zone, and wherein the fastening zone is directed towards the first face of the fixed portion of the bonding component. In many embodiments, the distal region of the bonding component fold-back portion includes a secondary fold-back portion. In many embodiments, the secondary fold-back portion includes a distal region, wherein the secondary fold-back portion distal region includes a first face and an oppositely directed second face, and wherein the fastening component is attached to the secondary fold-back portion distal region. In many embodiments, the fastening component is attached to the first face of the secondary fold-back portion distal region. In many embodiments, the fastening zone includes at least one of an adhesive and a plurality of hooks. In many embodiments, the fastening zone is removably attached to the first face of the fixed portion of the bonding component. In many embodiments, the fastening zone is attachable to a substrate. In many embodiments, the fastening zone is disposed on at least a portion of the second face of the distal region of the fold-back portion of the bonding component. In many embodiments, the fastening zone is disposed on at least a portion of the second face of the fastening component. In many embodiments, the fixation component includes a single bonding component having two fold-back portions. In many embodiments, the fixed portion and the fold-back portion are a single continuous piece. In many embodiments, the fixed portion and the fold-back portion are independent pieces connected to each other. In many embodiments, the bonding component includes a material selected from the group of a nonwoven, a nonwoven film laminate, a filmic construction, a woven, and any combination thereof. In many embodiments, disposed upon at least a portion of the first face of the fixed portion of the bonding component is at least one of a plurality of hooks and an adhesive. In many embodiments, the fastening component is connected to the fold-back portion of the bonding component by at least one of an adhesive and welding. In many embodiments, the bonding component comprises a fold-back portion, and wherein at least a portion of the bonding component fold-back portion is attached to the fastening component.

In many embodiments, fixation device further includes an extension system including at least one extension component including a first face and an oppositely directed second face, wherein the extension component is attached to at least one of the bonding component and the fastening component. In many embodiments, the fastening component includes at least one fold-back portion, and wherein at least a portion of the fold-back portion of the fastening component is attached to the extension component. In many embodiments, disposed on at least a portion of at least one of the first face of the extension component, the first face of the fixed portion of the bonding component, and the first face of the fastening component, is a release material. In many embodiments, disposed upon at least a portion of at least one of the second face of the fixed portion of bonding component, the second face of the extension component, and the second face of the fold-back portion of the bonding component, is an anti-flagging zone. In many embodiments, the anti-flagging zone includes at least one of an adhesive and a plurality of hooks. In many embodiments, the anti-flagging zone includes an adhesive. In many embodiments, the fixation system includes at least one of two bonding components, two fastening components and two extension components. In many embodiments, at least one of the members of the two bonding, the two fastening components and the two extension components, mirror each other, or substantially mirror each other.

Also described is an article including a substrate upon which is attached the fixation system described hereinabove. In many embodiments, the fastening zone attaches the fixation system to the substrate. In many embodiments, the substrate is one of a surface of a hybrid diaper insert pad or a surface of a hybrid diaper shell. In many embodiments, the hybrid diaper insert pad further comprises leg cuffs.

Further described is a hybrid diaper including a diaper shell and the hybrid diaper insert pad described hereinabove, wherein the fastening zone attaches the diaper shell to the fixation system.

Also described is a method of using the fixation system described hereinabove, the method including the steps of: providing the fixation system; adhering the second face of the bonding component fixed portion or the second face of the extension component to a first substrate; separating the fastening zone from the fixed portion of the bonding component, thereby exposing the fastening zone; and attaching the fastening zone to a second substrate. In many embodiments, the first substrate is a hybrid diaper insert pad. In many embodiments, the second substrate is a hybrid diaper shell. In many embodiments, the method further includes the steps of: detaching the fastening zone from the second substrate; exposing the anti-flagging zone; extending the fixation system around at least a portion of an article to which the fixation system is attached; and attaching at least one of the fastening zone and the anti-flagging zone to the first substrate.

Other features and advantages of the present invention will become apparent to those skilled in the art from the following detailed description. It is to be understood, however, that the detailed description of the various embodiments and specific examples, while indicating preferred and other embodiments of the present invention, are given by way of illustration and not limitation. Many changes and modifications within the scope of the present invention may be made without departing from the spirit thereof, and the invention includes all such modifications.

### BRIEF DESCRIPTION OF THE FIGURES

The accompanying drawings, which are included to provide a further understanding of the invention and are incorporated in and constitute a part of this specification, illustrate embodiments of the invention, and together with the description, serve to explain the principles of the invention. In the drawings:

FIG. 1 is a schematic illustration showing a fixation system including a bonding component, according to aspects described herein.

FIG. 2 is a schematic illustration showing a fixation system including a bonding component, according to aspects described herein.

FIG. 3A is a schematic illustration showing a fixation system including a bonding component and a fastening component, according to aspects described herein.

FIG. 3B is a schematic illustration showing a fixation system including a bonding component and a fastening component, according to aspects described herein.

FIG. 4 is a schematic illustration showing a fixation system including a bonding component and a fastening component, according to aspects described herein.

FIG. 5A is a schematic illustration showing a fixation system including a bonding component and a fastening component, according to aspects described herein.

FIG. 5B is a schematic illustration showing the fixation system of FIG. 5A in a partially extended position.

FIG. 5C is a schematic illustration showing the fixation system of FIGS. 5A and 5B in a fully extended position.

FIG. 6 is a schematic illustration showing a fixation system including a bonding component, a fastening component and an extension component, according to aspects described herein.

FIG. 7 is a schematic illustration showing a fixation system including a bonding component, a fastening component and an extension component, according to aspects described herein.

FIG. 8 is a schematic illustration showing a fixation system including a bonding component, a fastening component and an extension component, according to aspects described herein.

FIG. 9 is a schematic illustration showing a fixation system including a bonding component, a fastening component and an extension component, according to aspects described herein.

FIG. 10 is a schematic illustration showing a fixation system including a bonding component, a fastening component and an extension component, according to aspects described herein.

FIG. 11A is a schematic illustration showing a fixation system including a bonding component, a fastening component and an extension component, according to aspects described herein.

FIG. 11B is a schematic illustration showing the fixation system of FIG. 11A in a partially extended position.

FIG. 11C is a schematic illustration showing the fixation system of FIGS. 11A and 11B in an extended position.

FIG. 12 is a schematic illustration showing a fixation system including a bonding component and a fastening component, according to aspects described herein.

FIG. 13 is a schematic illustration showing a fixation system including a bonding component and a fastening component, according to aspects described herein.

None of the figures is drawn to scale.

### DETAILED DESCRIPTION

Aspects of the invention are disclosed in the following description and related drawings directed to specific embodiments of the disclosure. Alternate embodiments may be devised without departing from the spirit or the scope of the disclosure. Additionally, well-known elements of exemplary embodiments of the disclosure will not be described in detail or will be omitted so as not to obscure the relevant details of the disclosure.

As used herein, the word “exemplary” means “serving as an example, instance or illustration.” The embodiments described herein are not limiting, but rather are exemplary only. It should be understood that the described embodiments are not necessarily to be construed as preferred or advantageous over other embodiments. Moreover, the terms “embodiments of the disclosure”, “embodiments,” “disclosure” or “invention” do not require that all embodiments of the disclosure include the discussed feature, advantage or mode of operation.

As noted hereinabove, conventional hybrid diaper systems are associated with drawbacks such as handling complexity, lack of sustainability due to the presence of a liner that must be removed and disposed of, and adhesive residues remaining on the outer shell. The fixation system of the disclosure addresses at least one of these problems. Not only is the present fixation system liner-less and thus sustainable, it also addresses issues related to the storage and use of liner-less attachment systems. Disposable diaper pads are ordinarily stacked next to each other when stored in their packaging. If the adhesive used to attach the disposable diaper pad to the diaper shell remains exposed during storage, each of the diaper pads will undesirably adhere to its neighbour(s). This makes it difficult to separate the diaper pads, and also causes damage to the neighbouring diaper pad(s) when force is applied to a diaper pad to detach it from its neighbour. The disclosed fixation system protects the attachment adhesive until it is deliberately exposed in preparation for use of the diaper pad.

The fixation system includes at least one bonding component including at least one fixed portion and at least one fold-back portion. The fixed portion has a first face and an oppositely directed second face. Disposed upon at least a portion of the second face of the fixed portion of the bonding component is an adhesive. Thus, the fixed portion is attachable to a substrate, such as for example the surface of a disposable diaper insert or the surface of a hybrid diaper shell, via the adhesive. In some embodiments, the adhesive disposed on at least a portion of the second face of the fixed portion of the bonding component is attachable to the surface of a disposable diaper insert or the surface of a hybrid diaper shell. In some embodiments, the adhesive disposed on at least a portion of the second face of the fixed portion of the bonding component is attachable to the surface of a disposable diaper insert. In some embodiments, the adhesive disposed on at least a portion of the second face of the fixed portion of the bonding component is attachable to the surface of a hybrid diaper shell. In some embodiments, the adhesive layer on at least a portion of the second face of the fixed portion is continuous. In some embodiments, the adhesive layer on at least a portion of the second face of the fixed portion is discontinuous. Any suitable adhesive can be used, such as for example a non-pressure sensitive adhesive such as for example a hot melt adhesive or heat activated adhesive, or a pressure sensitive adhesive such as for example a hot melt rubber based solvent-based acrylic, water-based acrylic, or a solvent based rubber based pressure sensitive adhesive. In some embodiments, the adhesive on the second face of the fixed portion is a non-pressure sensitive adhesive. In some embodiments, the adhesive on the second face of the fixed portion is a pressure sensitive adhesive. In some embodiments, the fixed portion and the fold-back portion of the fixation system are a single continuous piece. In some embodiments, the fixed portion and the fold-back portion are separate pieces that are attached to each other. The fixed portion and the fold-back portion can be directly attached by any suitable means, such as for example by adhesion, lamination or welding. The fixed portion and fold-back portion of the bonding component can be indirectly attached by at least one additional component, such as for example, a U-bond. In some embodiments, the fold-back portion is a U-bond attached to the fixed portion of the bonding component. At each far end of the fixed portion of the bonding component is a distal region. The fold-back portion has a distal region toward the distal end of the fold-back portion, where the “distal end” is the end of the fold-back portion that lies furthest from the fixed portion of the bonding component. The distal region of the fold-back portion includes a first face and an oppositely directed second face. The fold-back portion of the bonding component is oriented so that at least a portion of the fold-back portion extends over at least a portion of the distal region of the fixed portion of the bonding component. The extension of the fold-back portion over at least a portion of the distal region of the fixed portion can be achieved by any suitable means, such as for example, by at least one fold in the bonding component. In some embodiments, the at least one fold is located at or near the point where the fixed portion of bonding component meets the fold-back portion of the bonding component. The at least one fold can have any suitable configuration. In some embodiments, the at least one fold is formed on a bonding component on which the fixed component and the fold-back portion are formed from a single continuous piece. In some embodiments the fixed portion and the fold-back portion are independent pieces that are attached to each other. In some embodiments, at least a portion of the fold-back portion lies parallel to or substantially parallel to the distal region of the fixed portion of the bonding component. This arrangement enables the fixation system to have at least one of a flatter profile or lower thickness.

The bonding component can be made of any suitable material, such as for example, a nonwoven, a nonwoven film laminate, a filmic construction, a woven, and any combination thereof. In all of the described embodiments, the bonding component can be any one of the described materials, or any other suitable material.

In some embodiments, the fixation system includes a single bonding component. In some embodiments, the single bonding component includes one fold-back portion at one end of the bonding component. In some embodiments, the single bonding component includes two fold-back portions, one at each end of the bonding component. In some embodiments, the fixation system includes more than one bonding component. In some embodiments, the fixation system includes two bonding components, namely a first bonding component and a second bonding component. In some embodiments, the first bonding component and the second bonding component are situated adjacent to each other. By “adjacent” is meant herein, next to or adjoining. In some embodiments there is a space between adjacent bonding components. In some embodiments there is no space between adjacent bonding components. In some embodiments, the first bonding component and the second bonding component are arranged so that they mirror, or substantially mirror each other. By “mirror” is meant herein that two components are identical, however, one of the components is oriented in the opposite direction to the other component, as would happen if one component were to be reflected in a mirror. The symmetry of mirrored first and second bonding components makes processing of the fixation system easier and more efficient, however, it will be understood that the first and second bonding components can be different from one another, and in some embodiments, they do not mirror each other.

The fixation system also includes at least one fastening zone. The at least one fastening zone is attachable to a substrate. Thus, the fastening zone can be used to attach the fixation system to a substrate, such as for example the shell of a hybrid diaper system or the surface of a hybrid insert pad. In some embodiments, the fastening zone is attachable to the surface of a disposable diaper insert or the surface of a hybrid diaper shell. In some embodiments, the fastening zone attachable to the surface of a disposable diaper insert. In some embodiments, the fastening zone is attachable to the surface of a hybrid diaper shell. The fastening zone can include any suitable material for attaching the fixation system to a substrate, for example, an adhesive, a plurality of hooks, or both. In some embodiments, the fastening zone includes an adhesive. In some embodiments, the fastening zone includes a plurality of hooks. The hooks can be attached by any suitable means. In some embodiments, the hooks are attached by one of ultrasonic sealing or welding, or extrusion lamination. In some embodiments, the fastening zone includes an adhesive and a plurality of hooks. Any suitable adhesive can be used for the fastening zone, such as, for example, the pressure sensitive and non-pressure sensitive adhesives described hereinabove.

The fixation system can have different modes, depending on the positioning, attachment or extension of different components of the fixation system. Different modes can be engaged, depending on whether the fastening zone is being protected before attachment, or exposed for attachment. The protection and exposure of the fastening zone can be achieved in a variety of ways. In some embodiments, the fixation system is in a protection mode. When the fixation system is in a “protection mode”, the fastening zone is oriented in such a manner that the fastening zone is protected from prematurely adhering to a substrate. In other words, the fastening zone is protected from adhering to a substrate until the fastening system has been prepared for attachment to a substrate. In some embodiments, when in the protection mode, the fastening zone is directed towards the first face of the fixed portion of the bonding component. In some embodiments, when in the protection mode, the fastening zone is removably attached to the first face of the fixed portion of the bonding component. In some embodiments, the fixation system is in a fastening mode. In the “fastening mode,” the fastening zone is exposed from its area of protection, and is thus prepared for attachment to a substrate. In some embodiments, when in the fastening mode, the fastening zone faces in the same direction as the first face of the fixed portion of the bonding component. In some embodiments, when in the fastening mode, the fastening zone is not attached to any component other than the component on which it resides.

The location of the fastening zone will depend on the configuration of the fixation system. In some embodiments, the at least one fastening zone is disposed on at least a portion of the distal region of the fold-back portion of the bonding component, or at least a portion of the fastening component. In some embodiments, the at least one fastening zone is disposed on at least one of, at least a portion of the distal region of the fold-back portion of the bonding component, and at least a portion of the fastening component.

In some embodiments, the fastening zone is disposed on at least a portion of the distal region of the fold-back portion of the bonding component. When the fastening system is in a protection mode, the fold-back portion of the bonding component is oriented so that at least a portion of the fold-back portion of the bonding component extends over at least a portion of the distal region of the fixed portion. In this orientation, the fastening zone is located on at least a portion of the second face of the distal region of the fold-back portion of the bonding component. Thus, the fastening zone is directed towards, or faces, the first face of the fixed portion of the bonding component. In this manner, the fastening zone is protected from prematurely adhering to a substrate. The area between the second face of the fold-back portion, and first face of the fixed portion of the bonding component, serves as an area of protection for the fastening zone. In some embodiments, in the protection mode, at least a portion of the fold-back portion lies parallel to or substantially parallel to the distal region of the fixed portion of the fixation system. In some embodiments, the fastening zone includes a plurality of hooks. In some embodiments, the bonding component is made of a nonwoven material, and the fastening zone includes a plurality of hooks. In some embodiments, the bonding component is made of a nonwoven material, and the fastening zone includes an adhesive and a plurality of hooks. The fastening zone hooks attach to the nonwoven material on the first face of the fixed component of the bonding component, thereby protecting the fastening zone while the fixation system is in the protection mode. As noted hereinabove, the bonding component is not restricted to a nonwoven material. In some embodiments, the bonding component is made of at least one of a nonwoven, a nonwoven film laminate, a filmic construction, a woven, and any combination thereof. In some embodiments, disposed on at least a portion of the first face of the fixed portion of the bonding component is an adhesive and a plurality of hooks. In some embodiments, the fastening zone includes an adhesive and a plurality of hooks, and disposed on at least a portion of the first face of the fixed portion of the bonding component is an adhesive and a plurality of hooks. The fastening zone hooks attach to the hooks on the first face of the fixed component, thereby protecting the fastening zone while the fixation system is in the protection mode. In some embodiments, the fastening zone includes an adhesive. In some embodiments, the fastening zone includes a plurality of hooks. In some embodiments, the fastening zone includes an adhesive and a plurality hooks. In some embodiments where the fastening zone includes an adhesive, or an adhesive and a plurality hooks, disposed on at least a portion of the first face of the fixed portion of the bonding component is a release material. Any suitable release material can be used, such as, for example, a silicone-based or fluor-based release material. The fastening zone adhesive lightly attaches to the release material, thereby protecting the fastening zone while the fixation system is in the protection mode. The release material enables the functional detachment of the fastening zone from the underlying layer, while also preventing damage to the underlying component caused by the force of removal of the fastening zone adhesive from the underlying layer, as well as preventing the undesirable flagging of the tape caused by premature detachment of the fastening zone from the underlying layer. When the fixation system is to be prepared for use, the fastening zone is exposed from its area of protection. This can be achieved, for example, by lifting the end of the fold-back portion of the bonding component, thereby separating the fastening zone from the fixed portion of the bonding component. With the fastening zone now exposed, the fixation system is in the fastening mode, where the fastening zone can be attached to a substrate, such as for example, the shell of a hybrid diaper system or the surface of a hybrid insert pad. In some embodiments, the fastening zone can be attached to the substrate by at least one of the fastening zone hooks and the adhesive. In some embodiments, the fastening zone can be attached to the substrate by the fastening zone hooks, or adhesive or both.

In some embodiments, at least a portion of the fold-back portion of the bonding component is adhesive free. In some embodiments, at least a portion of the second face of the distal region of the fold-back portion is adhesive free. In some embodiments, at least a portion of the fold-back portion of the bonding component is hook free. In some embodiments, at least a portion of the second face of the distal region of the fold-back portion of the bonding component is hook free. In some embodiments, at least a portion of the fold-back portion of the bonding component is at least one of hook free or adhesive free. In some embodiments, at least a portion of the second face of the distal region of the fold-back portion is at least one of adhesive free or hook free. In some embodiments, at least a portion of the fold-back portion of the bonding component is hook free and adhesive free. In some embodiments, at least a portion of the second face of the distal region of the fold-back portion is adhesive free and hook free.

In some embodiments, the fixation system includes at least one bonding component including at least one fixed portion including a first face and an oppositely directed second face, and at least one fold-back portion including a distal region. Further, in some of these embodiments, disposed upon at least a portion of the second face of the fixed portion may be an adhesive. The distal region of the fold-back portion may include a first face and an oppositely directed second face, and disposed on a portion of the distal region of the fold-back portion may be at least one fastening zone. The fastening zone may be attachable to a substrate, and may be directed towards the first face of the fixed portion of the bonding component.

In some embodiments, the fixation system includes at least one bonding component including at least one fixed portion including a first face and an oppositely directed second face, and at least one fold-back portion including a distal region. Further, in some of these embodiments, disposed on at least a portion of the second face of the fixed portion may be an adhesive. The distal region of the fold-back portion may include a first face and an oppositely directed second face. Disposed on a portion of the distal region of the fold-back portion may be at least one fastening zone that is attachable to a substrate. The fastening zone may be directed towards the first face of the fixed portion of the bonding component. The fastening zone can include an adhesive and a plurality of hooks. The bonding component can be made of at least one of a nonwoven, a nonwoven film laminate, a filmic construction, a woven, and any combination thereof.

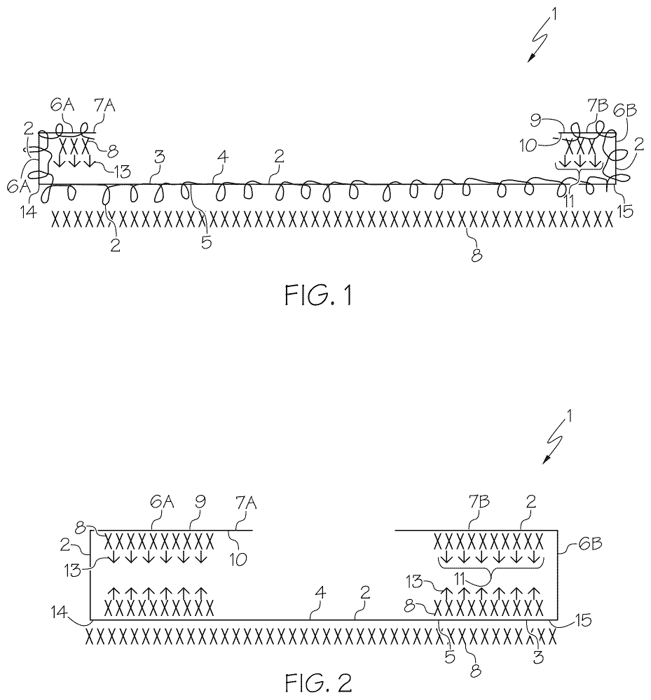

As exemplified in FIG. 1, in some embodiments, the fixation system 1 includes a bonding component 2 that includes a fixed portion 3 and two fold-back portions 6A and 6B. One fold-back portion 6A is located adjacent to the first distal region 14 of the fixed portion 3, and the other fold-back portion 6B is located adjacent to the second distal region 15 of the fixed portion 3. The fixed portion 3 and the fold-back portions 6A and 6B are a single continuous piece. The fixed portion 3 of the bonding component 2 includes a first face 4 and an oppositely directed second face 5. The fold-back portions 6A and 6B of the bonding component 2 each include a distal region 7A and 7B respectively. The distal regions 7A and 7B of each fold-back portion 6A and 6B each include a first face 9 and an oppositely directed second face 10. The distal region 7A and 7B of each fold-back portion 6A and 6B respectively lies parallel to the first distal region 14 and second distal region 15 respectively of the fixed portion 3. At least a portion of the second face 5 of the fixed portion 3 is coated with an adhesive 8. On a portion of each distal region 7A and 7B of the fold-back portions 6A and 6B respectively is at least one fastening zone 11. The bonding component 2 is made of a nonwoven material (visually represented as a line encircling a straight line). The fastening zone 11 includes an adhesive 8 and a plurality of hooks 13. The fastening zone 11 is directed towards the first face 4 of the fixed portion 3 of the bonding component 2, and the fastening zone 11 is removably attached to the fixed portion 3 of the bonding component 2 by the adhesive 8 and hooks 13 of the fastening zone 11. The fastening zone 11 is attachable to a substrate (not shown), such as, for example, the surface of a hybrid diaper insert pad, by the adhesive 8 and hooks 13 of the fastening zone 11. The adhesive 8 on the second face 5 of the bonding component 2 is attachable to a substrate (not shown), such as for example the surface of an insert pad for a hybrid diaper. In some embodiments, the adhesive 8 on the second face 5 of the bonding component 2 is removably attachable to a substrate (not shown), such as for example the surface of an insert pad for a hybrid diaper.

In some embodiments, the fixation system includes at least one bonding component including at least one fixed portion including a first face and an oppositely directed second face; and at least one fold-back portion including a distal region. Further, in some of these embodiments, disposed upon at least a portion of the second face of the fixed portion may be an adhesive. The distal region of the fold-back portion may include a first face and an oppositely directed second face, and disposed on a portion of the distal region of the fold-back portion, may be at least one fastening zone that is attachable to a substrate. The fastening zone may be directed towards the first face of the fixed portion of the bonding component, and the fastening zone can includes an adhesive and a plurality of hooks. Disposed on at least a portion of the first face of the fixed portion of the bonding component may be an adhesive and a plurality of hooks.

As exemplified in FIG. 2, in some embodiments, the fixation system 1 includes a bonding component 2 that includes a fixed portion 3 and two fold-back portions 6A and 6B. One fold-back portion 6A is located adjacent to the first distal region 14 of the fixed portion 3, and the other fold-back portion 6 is located adjacent to the second distal region 15 of the fixed portion 3. The fixed portion 3 and the fold-back portions 6A and 6B are a single continuous piece. The fixed portion 3 of the bonding component 2 includes a first face 4 and an oppositely directed second face 5. Each fold-back portion 6A and 6B of the bonding component 2 includes a distal region 7A and 7B respectively. The distal regions 7A and 7B of the fold-back portions 6A and 6B each includes a first face 9 and an oppositely directed second face 10. The distal region 7A and 7B of each fold-back portion 6A and 6B respectively lies parallel to the first distal region 14 and second distal region 15 respectively of the fixed portion 3. At least a portion of the second face 5 of the fixed portion 3 is coated with an adhesive 8. On a portion of the distal regions 7A and 7B of each fold-back portion 6A and 6B respectively is at least one fastening zone 11. Disposed on at least a portion of the first face 4 of the fixed portion 3 of the bonding component 2 is an adhesive 8 and a plurality of hooks 13. The fastening zone 11 includes an adhesive 8 and a plurality of hooks 13. The fastening zone 11 is directed towards the first face 4 of the fixed portion 3 of the bonding component 2, so that the adhesive 8 and hooks 13 of the fastening zone 11 reside above the adhesive 8 and hooks 13 on the first face 4 of the fixed portion 3 of the bonding component 2. The adhesive 8 and hooks 13 on the fixed portion 3 of the bonding component 2 and on the fastening zone 11, attach the fastening zone 11 to the fixed portion 3 of the bonding component 2. The fastening zone 11 is removably attachable to a substrate (not shown), such as for example the surface of a hybrid diaper outer shell, by the adhesive 8 and hooks 13 of the fastening zone 11. The adhesive 8 on the second face 5 of the bonding component 2 is attachable to a substrate (not shown), such as for example the surface of a hybrid diaper insert pad. In some embodiments, the adhesive 8 on the second face 5 of the bonding component 2 is removably attachable to a substrate (not shown), such as for example the surface of a hybrid diaper insert pad.

The fixation system can also include at least one fastening component. In some embodiments, the fixation system optionally includes at least one fastening component connected to the distal region of the fold-back portion of the bonding component. In some embodiments, the fixation system includes at least one fastening component attached to the distal region of the fold-back portion of the bonding component. The fastening component can be attached or connected to the fold-back portion of the bonding component by any suitable means, such as for example by folding, adhesion, lamination or welding. In some embodiments, the fastening component is attached to the fold-back portion of the bonding component by an adhesive. The fastening component includes a first face and an oppositely directed second face. The presence of the fastening component on the fixation system enables easier manufacturing of the fixation system, especially when the fixation system has a wide fold-back portion. In some embodiments, the fastening zone is disposed on at least a portion of the fastening component. In some embodiments, the fastening zone is disposed on at least a portion of second face of the fastening component.

As noted herein-above, the fixation system can have different modes, depending on the positioning, attachment or extension of different components of the fixation system. In some embodiments, when the fastening system is in a protection mode, the fastening component is oriented so that at least a portion of the fastening component extends over at least a portion of the distal region of the fixed portion of the bonding component. In this orientation, the fastening zone is directed towards the first face of the fixed portion of the bonding component. In this manner, the fastening zone is protected from prematurely adhering to a substrate. The area between the second face of fastening component, and first face of the fixed portion of the bonding component, serves as an area of protection for the fastening zone. In some embodiments, in the protection mode, at least a portion of the fastening component lies parallel to or substantially parallel to the distal region of the fixed portion of the fixation system. In some embodiments, when in the protection mode, the fastening zone is removably attached to the first face of the fixed portion of the bonding component. In some embodiments, when in the fastening mode, the fastening zone faces in the same direction as the first face of the fixed portion of the bonding component. In some embodiments, when in the fastening mode, the fastening zone is not attached to any component other than the component on which it resides, such as for example the fastening component. In some embodiments, the bonding component is made of a nonwoven material, and the fastening zone includes a plurality of hooks. In some embodiments, the bonding component is made of a nonwoven material, and the fastening zone includes an adhesive and a plurality of hooks. The fastening zone hooks attach to the nonwoven material on the first face of the fixed component of the bonding component, thereby protecting the fastening zone while the fixation system is in the protection mode. As noted hereinabove, the bonding component is not restricted to a nonwoven material. In some embodiments, the bonding component is made of at least one of a nonwoven, a nonwoven film laminate, a filmic construction, a woven, and any combination thereof. In some embodiments, the fastening zone includes a plurality of hooks, and disposed on at least a portion of the first face of the fixed portion of the bonding component is a plurality of hooks. In some embodiments, the fastening zone includes an adhesive and a plurality of hooks, and disposed on at least a portion of the first face of the fixed portion of the bonding component is an adhesive and a plurality of hooks. The fastening zone hooks attach to the hooks on the first face of the fixed component, thereby protecting the fastening zone while the fixation system is in the protection mode. In some embodiments, the fastening zone includes an adhesive. In some embodiments, the fastening zone includes a plurality of hooks. In some embodiments, the fastening zone includes an adhesive and a plurality hooks. In some embodiments the fastening zone includes an adhesive, or an adhesive and a plurality hooks, and disposed on at least a portion of the first face of the fixed portion of the bonding component is a release material. Any suitable release material can be used, such as, for example, a silicone-based or fluor-based release material. The fastening zone adhesive lightly attaches to the release material, thereby protecting the fastening zone while the fixation system is in the protection mode. The release material enables the functional detachment of the fastening zone from the underlying layer, while also preventing damage to the underlying component caused by the force of detachment of the fastening zone adhesive from the underlying layer, and preventing the undesirable flagging of the tape caused by premature removal of the fastening zone from the underlying layer. When the fixation system is to be prepared for use, the fastening zone is exposed from its area of protection. This can be achieved, for example, by separating the fastening zone from the fixed portion of the bonding component. With the fastening zone now exposed, the fixation system is in the fastening mode, where the fastening zone can be attached to a substrate, such as for example, a hybrid diaper shell. In some embodiments, the fastening zone can be attached to the substrate by at least one of the fastening zone hooks and adhesive. In some embodiments, the fastening zone can be attached to the substrate by the fastening zone hooks, adhesive, or both.

In some embodiments, the fixation system includes a single bonding component. In some embodiments, the single bonding component includes a single fastening component. In some embodiments, the single bonding component includes two fastening components, each of which is attached to a respective fold-back portion located at opposite ends of the fixed portion of the bonding component. In some embodiments, the fixation system includes more than one bonding component. In some embodiments, the fixation system includes two bonding components. In some embodiments, the first bonding component and the second bonding component are situated adjacent to each other. In some embodiments, the first bonding component and the second bonding component are arranged so that they mirror, or substantially mirror each other. In some embodiments, the bonding components do not mirror each other.

In some embodiments, at least a portion of the fastening component is adhesive free. In some embodiments, at least a portion of the second face of the fastening component is adhesive free. In some embodiments, at least a portion of the fastening component is hook free. In some embodiments, at least a portion of the second face of the fastening component is hook free. In some embodiments, at least a portion of the fastening component is at least one of hook free and adhesive free. In some embodiments, at least a portion of the second face of the fastening component is at least one of adhesive free and hook free. In some embodiments, at least a portion of the fastening component is hook free and adhesive free. In some embodiments, at least a portion of the second face of the fastening component is adhesive free and hook free.

In some embodiments, the fixation system includes at least one bonding component including at least one fixed portion including a first face and an oppositely directed second face, and at least one fold-back portion including a distal region. Further, in some of these embodiments, the fixation system may further include at least one fastening component connected to the distal region of the bonding component fold-back portion. Disposed upon at least a portion of the second face of the fixed portion may be an adhesive. The distal region of the fold-back portion may include a first face and an oppositely directed second face. The fastening component may include a first face and an oppositely directed second face. Disposed on at least a portion of the fastening component may be at least one fastening zone that is attachable to a substrate. The fastening zone may be directed towards the first face of the fixed portion of the bonding component.

In some embodiments, the fixation system includes at least one bonding component including at least one fixed portion including a first face and an oppositely directed second face, and at least one fold-back portion including a distal region; and optionally at least one fastening component connected to the distal region of the bonding component fold-back portion. Further, in some of these embodiments, disposed upon at least a portion of the second face of the fixed portion may be an adhesive. The distal region of the fold-back portion may include a first face and an oppositely directed second face. The optional fastening component may include a first face and an oppositely directed second face. Disposed on at least a portion of the fastening component, may be at least one fastening zone that is attachable to a substrate. The fastening zone may be directed towards the first face of the fixed portion of the bonding component.

In some embodiments, the fixation system includes at least one bonding component including at least one fixed portion including a first face and an oppositely directed second face, and at least one fold-back portion including a distal region; and optionally at least one fastening component connected to the distal region of the bonding component fold-back portion. Further, in some of these embodiments, disposed upon at least a portion of the second face of the fixed portion may be an adhesive. The distal region of the fold-back portion may include a first face and an oppositely directed second face. The optional fastening component may include a first face and an oppositely directed second face. Disposed on at least a portion of the fastening component, may be at least one fastening zone that is attachable to a substrate. The fastening zone may be directed towards the first face of the fixed portion of the bonding component. The bonding component can be made of a material selected from at least one of a nonwoven, a nonwoven film laminate, a filmic construction, a woven, or any combination thereof. The fastening zone may include at least one of an adhesive and a plurality of hooks.

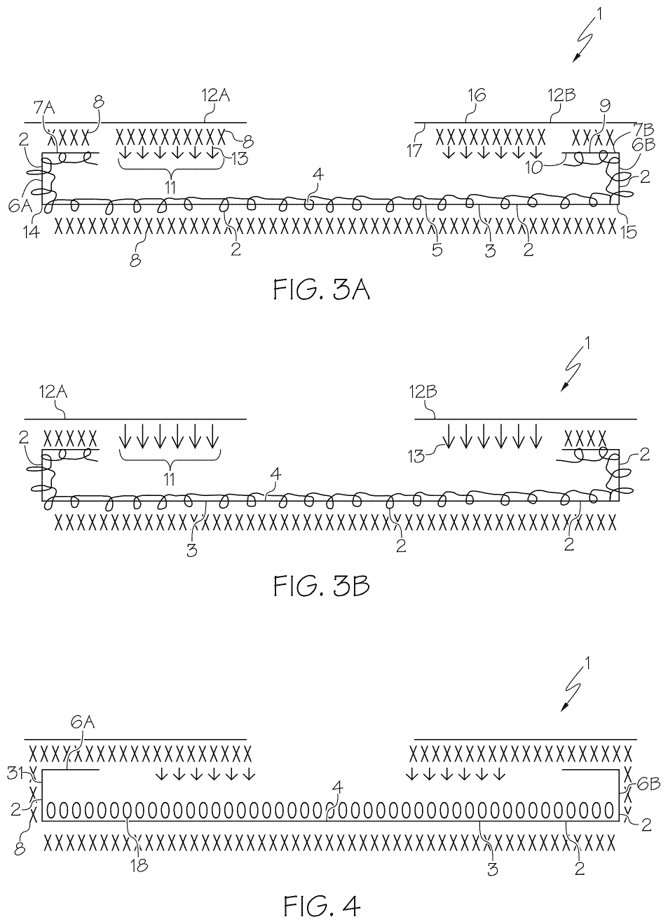

As exemplified in FIG. 3A, in some embodiments, the fixation system 1 includes a bonding component 2, that includes a fixed portion 3 and two fold-back portions 6A and 6B. The bonding component 2 is made of a nonwoven material (visually represented as a line encircling a straight line). One fold-back portion 6A is located adjacent to the first distal end 14 of the fixed portion 3, and the other fold-back portion 6B is located adjacent to the second distal end 15 of the fixed portion 3. The fixed portion 3 and the fold-back portions 6A and 6B are a single continuous piece. The fold-back portions 6A and 6B of the bonding component 2 each include a distal regions 7A and 7B respectively. The distal region 7A and 7B of each fold-back portion 6A and 6B respectively lies parallel to the first distal region 14 and second distal region 15 respectively of the fixed portion 3. A fastening component 12A and 12B is attached to the distal region 7A and 7B respectively of each of the bonding component fold-back portions 6A and 6B by an adhesive 8. Each fastening component 12A and 12B includes a first face 16 and an oppositely directed second face 17. The fixed portion 3 of the bonding component 2 includes a first face 4 and an oppositely directed second face 5. The distal region 7A and 7B of each fold-back portion 6A and 6B respectively includes a first face 9 and an oppositely directed second face 10. At least a portion of the second face 5 of each fixed portion 3 is coated with an adhesive 8. On a portion of each fastening component 12A and 12B is a fastening zone 11. The fastening zone 11 includes an adhesive 8 and a plurality of hooks 13. The fastening zone 11 is directed towards the first face 4 of the fixed portion 3 of the bonding component 2, and the fastening zone 11 is attached to the fixed portion 3 of the bonding component 2 by the adhesive 8 and hooks 13 of the fastening zone 11. The fastening zone 11 is attachable to a substrate (not shown), such as for example the surface of a hybrid diaper outer shell, by the adhesive 8 and hooks 13 of the fastening zone 11. The adhesive 8 on the second face 5 of the bonding component 2 is attachable to a substrate (not shown), such as for example the surface of a hybrid diaper insert pad.

FIG. 3B shows a fixation system 1 similar to the fixation system exemplified in FIG, 3A, where rather than an adhesive and a plurality hooks, the fastening zone 11 on the fastening components 12A and 12B include only a plurality of hooks 13. The fastening zone 11 is directed towards the first face 4 of the fixed portion 3 of the bonding component 2, and the fastening zone 11 is attached to the fixed portion 3 of the bonding component 2 by the hooks 13 of the fastening zone 11. The hooks 13 can be attached to the fastening component 12 by any suitable means. In some embodiments, the hooks 13 are attached by one of ultrasonic sealing or welding, and extrusion lamination.

As noted herein-above, in some embodiments, a release material is deposited on at least a portion of the first face of the bonding component.

In some embodiments, the fixation system includes at least one bonding component including at least one fixed portion including a first face and an oppositely directed second face, and at least one fold-back portion including a distal region; and at least one fastening component connected to the distal region of the bonding component fold-back portion. Further, in some of these embodiments, disposed upon at least a portion of the second face of the fixed portion may be an adhesive. The distal region of the fold-back portion may include a first face and an oppositely directed second face. The fastening component may include a first face and an oppositely directed second face. Disposed on a portion of the distal region of the fold-back portion, may be at least one fastening zone that is attachable to a substrate. The fastening zone may be directed towards the first face of the fixed portion of the bonding component. The fastening zone may include an adhesive and a plurality of hooks. Disposed on at least a portion of the first face of the fixed portion of the bonding component may be a release material.

FIG. 4 shows a fixation system 1 similar to the fixation system exemplified in FIG, 3A, where the bonding component can be made of a nonwoven, a nonwoven film laminate, a filmic construction, a woven, and any combination thereof, and disposed on the first face 4 of the fixed portion 3 of the bonding component 2 is a layer of a release material 18. At least a portion of the fold-back portions 6A and 6B of the bonding component 2 have an adhesive 8 disposed on the outwardly facing edges 31 of the fold-back portions 6A and 6B.

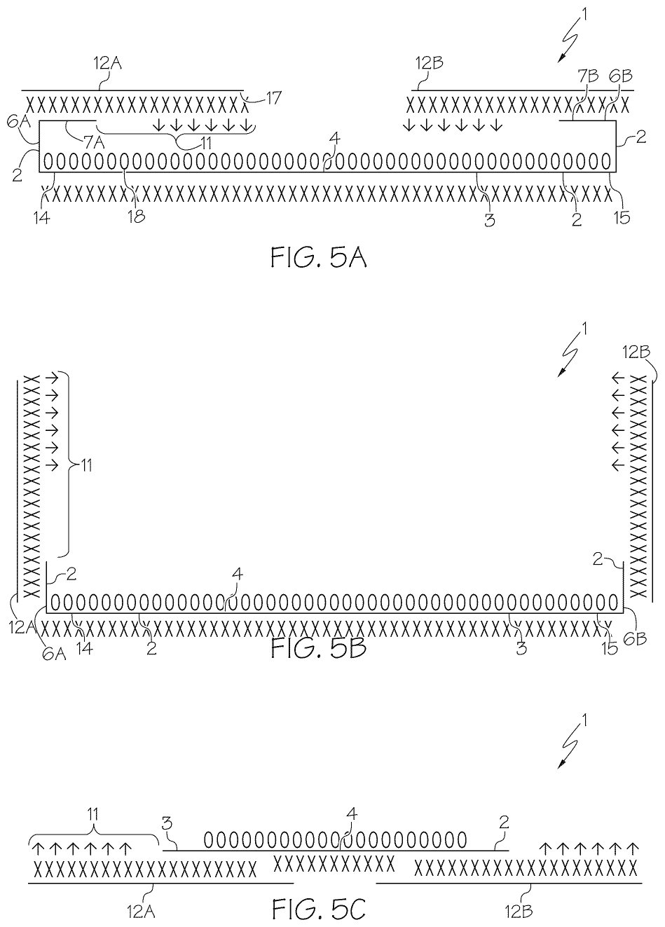

FIGS. 5A-5C show a fixation system in different configurations or modes. FIG. 5A shows a fixation system 1 that is similar to the fixation system exemplified in FIG, 3A, where the bonding component 2 is made of a material that is not a non-woven; disposed on the first face 4 of the fixed portion 3 of the bonding component 2 is a layer of a release material 18; and the fixation system 1 is in a protection mode, with each fastening zone 11 protected between the second face 17 of the fastening components 12A and 12B, and the first face 4 of the fixed portion 3 of the bonding component 2. The distal region 7A and 7B of each fold-back portion 6A and 6B respectively lies parallel to the first distal region 14 and second distal region 15 respectively of the fixed portion 3. FIG. 5B shows the fixation system 1 of FIG. 5A, with the fastening zone 11 having been separated from the first face 4 of the fixed portion 3 of the bonding component 2. In this configuration, the fold-back portions 6A and 6B of the bonding component 2 do not lie parallel to or substantially parallel to the distal regions 14 and 15 of the fixed portion 3 of the bonding component 2. Likewise, the fastening components 12A and 12B do not lie parallel to the fixed portion 3 of the bonding component 2. The fixation system is in the fastening mode, ready for attachment to a substrate. FIG. 5C shows the fixation systems 1 of FIGS. 5A and 5B with the bonding component 2 fully extended so that the fastening components 12A and 12B are parallel to and in substantially the same plane as the bonding component 2, such that the fastening zone 11 is facing in the same direction as the first face 4 of the fixed portion 3 of the bonding component 2. The fixation system remains in the fastening mode, ready for attachment to a substrate.

In some embodiments, the fixation system further includes an extension system. The extension system enables the tape length to be extended, typically after the fastening zone has been detached from a substrate. This feature is useful, for example, to prepare an article to which the fixation system is attached, for disposal. For example, the extension system enables a user to secure a hybrid diaper insert pad for disposal, as the extension system enables the fixation system to be extended around at least a portion of the insert pad to which the fixation system is attached, and thereby contain any soiling within the insert pad. In some embodiments, the extension system includes at least one extension component including a first face and an oppositely directed second face. The extension component can be attached to any suitable location on the fixation system, such as for example it can be attached to the bonding component or the fastening component. In some embodiments, at least a portion of the extension component is attached to the bonding component of the fixation system. In some embodiments, at least a portion of the extension component is attached to the fastening component of the fixation system. In some embodiments, at least a portion of the extension component is attached to one of the fastening component and the bonding component of the fixation system. The extension component can be attached to the bonding component or the fastening component of the fixation system by any suitable means, such as for example adhesion, lamination or welding. In some embodiments the extension component is attached to the bonding component or the fastening component by adhesion.

In some embodiments, the extension component is configured for attachment to the fixation system. In some embodiments, the extension component includes at least one fold-back portion. In some embodiments, at least a portion of the extension component fold-back portion is attached to the bonding component. The extension component fold-back portion can be attached to any suitable location on the bonding component. In some embodiments, extension component fold-back portion is attached to the second face of the fixed component of the bonding component.

In some embodiments, the extension component is attached to the fastening component. In some embodiments, the fastening component includes a fold-back portion. In some embodiments, the fastening component includes a fold-back portion, and at least a portion of the fastening component fold-back portion is attached to the extension component. The fastening component fold-back portion can be attached to any suitable location on the fastening component. In some embodiments, fastening component fold-back portion is attached to the second face of the extension component.

In some embodiments, disposed upon at least a portion of the second face of the fixed portion of the bonding component is an adhesive. In some embodiments, at least a portion of the second face of the fixed portion is attachable to a substrate, such as for example the surface of a disposable diaper insert, via the adhesive. Any suitable adhesive can be used, such as for example a non-pressure sensitive adhesive such as for example a hot melt adhesive or heat activated adhesive, or a pressure sensitive adhesive such as for example a hot melt rubber based solvent-based acrylic, water-based acrylic, or a solvent based rubber based pressure sensitive adhesive.

In some embodiments, the extension system includes one extension component. In some embodiments, the extension system includes more than one extension component. In some embodiments, the extension system includes two extension components. In some embodiments, the first extension component and the second extension component are situated adjacent to each other. In some embodiments, the first extension component and the second extension component are arranged so that they mirror, or substantially mirror each other. In some embodiments, the extension components do not mirror each other.

In some embodiments, the fixation system includes one bonding component and one extension component. In some embodiments, the fixation system includes one bonding component and more than one extension component. In some embodiments, the fixation system includes one bonding component and two extension components. In some embodiments, the fixation system includes more than one bonding component and more than one extension component. In some embodiments, the fixation system includes two bonding components and two extension components. To reduce the cost of materials, in some embodiments, the fixation system includes a single bonding component including two fold-back portions, and one extension component.

In some embodiments, the fixation system includes at least one anti-flagging zone. The anti-flagging zone helps to keep two fixation system components temporarily attached. The temporarily attached components will remain attached until the two components are deliberately separated. Thus, the anti-flagging zone prevents the tape from flagging. By “flagging” is meant prematurely separating, for example during conversion, storage or use. An anti-flagging zone can be disposed on a face of any of the components of the fixation system. In some embodiments, an anti-flagging zone is deposited on at least a portion of at least one of the second face of the fixed portion of bonding component, the second face of the extension component, or the second face of the fold-back portion of the bonding component. In some embodiments, the anti-flagging zone keeps at least one of the extension component and the fastening component, the bonding component and the extension component, or the bonding component and the fastening component, attached during storage and attachment of the fastening component to a substrate. When desired, for example when the fixation system is to be prepared for disposal of an article such as for example, the diaper insert pad, the fastening zone can be detached from the substrate to which it had been attached. The fixation system can be fully extended, or substantially fully extended, exposing the anti-flagging zone. With the tape fully extended, or substantially fully extended, the tape can be extended around at least a portion of the article to which the fixation system is attached, and the anti-flagging zone or fastening zone can be attached to the substrate or article, for example, for disposal. When the fastening zone is attached to the substrate or article in this manner, at least a portion of the extension component can be twisted to position the fastening zone so that it can be attached to the article. In some embodiments, the anti-flagging zone includes an adhesive or a plurality of hooks, or both. In some embodiments, the anti-flagging zone includes an adhesive.

The anti-flagging zone can be located on any suitable location on the fixation system. Depending on the configuration of the fixation system, in some embodiments, the anti-flagging zone can be located on the extension component or the bonding component. The anti-flagging zone is attachable to a substrate. Thus, the anti-flagging zone can be used to attach the extension component to a substrate, such as for example the outer surface of a hybrid diaper insert pad. The anti-flagging zone can include any suitable material for attaching the extension component to a substrate. In some embodiments, the anti-flagging zone includes an adhesive. Any suitable adhesive can be used for the fastening zone, such as, for example, those described hereinabove.

In some embodiments, the fixation system includes at least one bonding component including at least one fixed portion including a first face and an oppositely directed second face, and at least one fold-back portion including a distal region; at least one fastening component attached to the distal region of the bonding component fold-back portion; and at least one extension component. Further, in some of these embodiments, disposed upon at least a portion of the second face of the fixed portion of the bonding component may be an adhesive. The distal region of the fold-back portion of the bonding component may include a first face and an oppositely directed second face. The fastening component may include a first face and an oppositely directed second face. Disposed on at least a portion of the second face of the fastening component may be at least one fastening zone that is directed towards the first face of the fixed portion of the bonding component. The extension component may include a first face and an oppositely directed second face. In some embodiments, at least a portion of the bonding component fold-back portion is attached to the fastening component. In some embodiments, at least a portion of the extension component is attached to the bonding component of the fixation system. In some embodiments, the extension component includes a fold-back portion including a distal region, and at least a portion of the extension component fold-back portion is attached to the at least a portion of the second face of the bonding component. In some embodiments, at least a portion of the distal region of the extension component fold-back portion is attached to the at least a portion of the second face of the bonding component. In some embodiments, at least a portion of the extension component is attached to the fastening component of the fixation system. In some embodiments, the fastening component includes a fold-back portion including a distal region, and at least a portion of the fastening component fold-back portion is attached to the at least a portion of the second face of the extension component. In some embodiments, at least a portion of distal region of the fastening component fold-back portion is attached to the at least a portion of the second face of the extension component. In some embodiments, at least a portion of the extension component is attached to one of the fastening component or the bonding component of the fixation system. In some embodiments, at least a portion of the extension component is attached to the fastening component or the bonding component.

In some embodiments, an anti-flagging zone is disposed on at least a portion of the extension component. In some embodiments, when the fixation system is in a protection mode, the extension component is oriented so that at least a portion of extension component extends over at least a portion of the fastening component. In this orientation, the anti-flagging zone is located on at least a portion of the second face of the extension component. Thus, the anti-flagging zone is directed towards the first face of the fastening component. In this manner, the anti-flagging zone is protected from prematurely adhering to a substrate. The area between the second face of the extension component, and the first face of the fastening component, serves as an area of protection for the anti-flagging zone. In some embodiments, in the protection mode, at least a portion of the extension component lies parallel to or substantially parallel to the fastening component. The adhesive in the anti-flagging zone attaches the second face of the extension component to the first face of the fastening component, thereby protecting the anti-flagging zone while the fixation system is in the protection mode. In some embodiments, disposed on at least a portion of the first face of the fastening component is a release material. Any suitable release material can be used, such as, for example, a silicone-based or fluor-based release material. The anti-flagging zone adhesive lightly attaches to the release material while the fixation system is in the protection mode, thereby enabling the functional removal of the anti-flagging zone from the underlying first face of the fastening component, while also preventing damage to the underlying fastening component caused by the force of removal of the anti-flagging zone adhesive from the underlying layer, and preventing the undesirable flagging of the tape caused by premature removal of the anti-flagging zone from the underlying layer. When the fixation system is to be prepared for use, the fastening zone is exposed from its area of protection. This can be achieved by separating the fastening zone from the fixed portion of the bonding component. With the fastening zone now exposed, the fixation system is in the fastening mode, where the fastening zone can be attached to a substrate, such as for example, a hybrid diaper shell. The fastening zone can be attached to the substrate by at least one of the fastening zone hooks and adhesive. The anti-flagging-zone remains protected during the fastening mode, as the extension component remains attached to the fastening component. When the fixation system is to be prepared for disposal, the fastening zone is detached from the surface to which it had been attached, and the anti-flagging zone is exposed from its area of protection. This can be achieved by separating the anti-flagging zone from the fastening component. With the anti-flagging zone now exposed, the fixation system is in the extension mode, where the anti-flagging zone can be attached to a substrate, such as for example, the outer surface of a hybrid diaper insert pad.

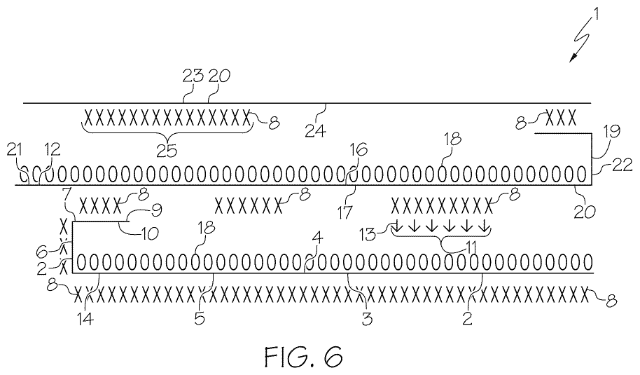

As exemplified in FIG. 6, in some embodiments, the fixation system 1 includes a bonding component 2, that includes a fixed portion 3 and a fold-back portion 6; a fastening component 12, and an extension component 20. The bonding component's 2 fixed portion 3 and the bonding component's 2 fold-back portion 6 are a single continuous piece. The distal region 7 of the fold-back portion 6 of the bonding component 2 lies parallel to the first distal region 14 of the fixed portion 3 of the bonding component 2. Disposed on the first face 4 of the bonding component 2 is a release material 18. The fastening component 12 is connected to the distal region 7 of the bonding component 2 fold-back portion 6 by an adhesive 8. The fastening component 12 includes a first face 16 and an oppositely directed second face 17, a first distal region 21 and a second distal region 22, and a fold-back portion 19 in the second distal region 22. Disposed on the first face 16 of the fastening component 12 is a release material 18. The extension component 20 is connected to the fastening component fold-back portion 19 by an adhesive 8. The fixed portion 3 of the bonding component 2 includes a first face 4 and an oppositely directed second face 5. The distal region 7 of the fold-back portion 6 on the bonding component 2 includes a first face 9 and an oppositely directed second face 10. At least a portion of the second face 5 of the fixed portion 3 is coated with an adhesive 8. The second face 5 of the fixed portion 3 can be fully coated with an adhesive 8. It will be understood that at least a portion of the second face 5 of the fixed portion 3 can be adhesive free. Disposed on at least a portion of the second face 17 of the fastening component 12 is a fastening zone 11. The fastening zone 11 includes an adhesive 8 and a plurality of hooks 13. The fastening zone 11 is directed towards the first face 4 of the fixed portion 3 of the bonding component 2, and the fastening zone 11 is attached to the fixed portion 3 of the bonding component 2 by the adhesive 8 and hooks 13 of the fastening zone 11, thus attaching the fastening component 12 to the bonding component 2. Optionally, the fastening component 12 can be further attached to the bonding component 2 by an adhesive region 8 on the second face 17 of the fastening component 12, located between the fastening zone 11 and the point of attachment of the fastening component 12 to the distal region 7 of the bonding component 2 fold-back portion 6. The fastening zone 11 is attachable to a substrate (not shown), such as for example the surface of a hybrid diaper insert pad or the surface of a hybrid diaper shell, by the adhesive 8 and hooks 13 of the fastening zone 11. The adhesive 8 on the second face 5 of the bonding component 2 is attachable to a substrate (not shown), such as for example the surface of a hybrid diaper insert pad. Disposed on a portion of the first face 16 of the fastening component 12 is a release material 18. The extension component 20 includes a first face 23 and a second face 24. Disposed on a portion of the second face 24 of the extension component 20 is an anti-flagging zone 25 that includes an adhesive 8. The anti-flagging zone 25 is directed towards the first face 16 of the fastening component 12, and the anti-flagging zone 25 is attached to the first face 16 of the fastening component 12 by the adhesive 8 of the anti-flagging zone 25. The anti-flagging zone 25 is attachable to a substrate (not shown), such as for example the surface of a hybrid diaper insert pad.

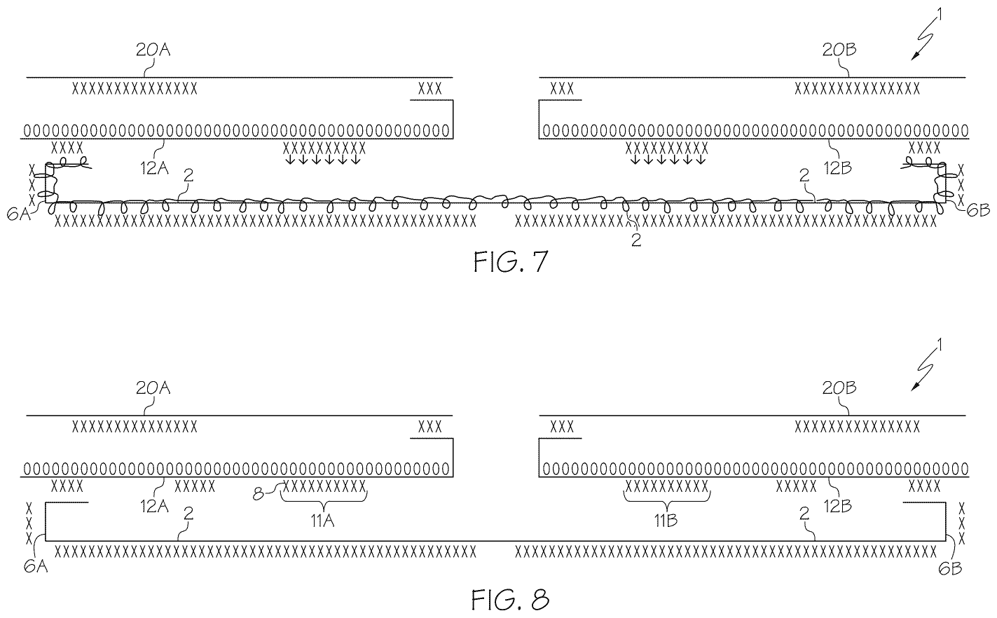

As described hereinabove, in some embodiments, at least one portion of the fixation system mirrors at least one other portion of the fixation system. Any suitable portion of the fixation system can mirror any other portion of the fixation system. FIG. 7 exemplifies some embodiments where a fixation system, similar to that of FIG. 6, includes some mirrored components. In FIG. 7, the bonding component 2 is made of a nonwoven material (visually represented as a line encircling a straight line) with no release material disposed thereupon, and the fixation system 1 includes a mirrored bonding component 2, fastening components 12A and 12B and extension components 20A and 20B. The mirrored bonding component 2 is a single component having two mirrored fold-back portions 6A and 6B; while the fixation system 1 includes two independent mirrored fastening components 12A and 12B; and two independent mirrored extension components 20A and 20B. Each side of the fixation system is identical to the other side, but reversed, as is reflected in a mirror.

It will be understood that there are embodiments where each side of the fixation system is not identical to the other, whether in dimension, arrangement of the components, or even presence of certain components. For example, in some embodiments, at least one component on one side of the fixation system can be shorter than the corresponding component on the other side of the fixation system. In some embodiments, at least one component on one side of the fixation system can be longer than the corresponding component on the other side of the fixation system. In some embodiments, one side of fixation system can includes an extension component, while the other side does not.

FIG. 8 exemplifies some embodiments where a fixation system, similar to that of FIG. 6, includes some mirrored components. In FIG. 8, rather than an adhesive and hook fastening zone, the fastening zones 11A and 11B each include an adhesive 8, and the fixation system 1 includes a mirrored bonding component 2, fastening components 12A and 12B and extension components 20A and 20B. The mirrored bonding component 2 is a single component having two mirrored fold-back portions 6A and 6B; while the fixation system 1 includes two independent mirrored fastening components 12A and 12B and two independent mirrored extension components 20A and 20B. Each side of the fixation system 1 is identical to the other side, but reversed, as if reflected in a mirror.

In some embodiments, an anti-flagging zone is disposed on at least a portion of the fixed portion of the bonding component. In some embodiments, when the fixation system is in a protection mode, the extension component is oriented so that at least a portion of bonding component extends over at least a portion of the extension component. In this orientation, the anti-flagging zone is located on at least a portion of the second face of the fixed portion of the bonding component. Thus, the anti-flagging zone is directed towards the first face of the extension component. In this manner, the anti-flagging zone is protected from prematurely adhering to a substrate. The area between the second face of the bonding component, and the first face of the extension component, serves as an area of protection for the anti-flagging zone. In some embodiments, in the protection mode, the extension component lies parallel to or substantially parallel to the bonding component. The adhesive in the anti-flagging zone attaches the second face of the fixed portion of the bonding component to the first face of the extension component, thereby protecting the anti-flagging zone while the fixation system is in the protection mode. In some embodiments, disposed on at least a portion of the first face of the extension component is a release material. Any suitable release material can be used, such as, for example, a silicone-based or fluor-based release material. The anti-flagging zone adhesive lightly attaches to the release material while the fixation system is in the protection mode, thus enabling the functional removal of the anti-flagging zone from the underlying extension component, while also preventing damage to the underlying extension component caused by the force of removal of the anti-flagging zone adhesive from the underlying layer, and preventing the undesirable flagging of the tape caused by premature removal of the anti-flagging zone from the underlying layer. When the fixation system is to be prepared for use, the fastening zone is exposed from its area of protection. This can be achieved by separating the fastening zone from the fixed portion of the bonding component. With the fastening zone now exposed, the fixation system is in the fastening mode, where the fastening zone can be attached to a substrate, such as for example, a hybrid diaper shell. The fastening zone can be attached to the substrate by at least one of the fastening zone hooks or adhesive. The anti-flagging zone remains protected during the fastening mode, as the extension component remains attached to the bonding component. When the fixation system is to be prepared for disposal of the article to which it is attached, the fastening zone is detached from the surface to which it had been attached, such as for example the shell of a hybrid diaper, and the anti-flagging zone is exposed from its area of protection. This can be achieved by separating the anti-flagging zone from the extension component. With the anti-flagging zone now exposed, the fixation system is in the extension mode, where the anti-flagging zone can be attached to a substrate, such as for example, the outer surface of a hybrid diaper insert pad.

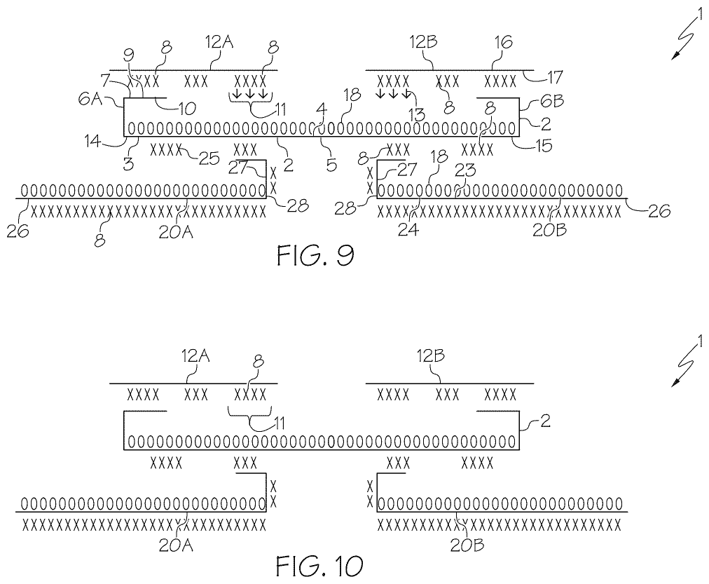

As exemplified in FIG. 9, in some embodiments, the fixation system 1 includes a bonding component 2 that includes a fixed portion 3 and two fold-back portions 6A and 6B. The fixed portion 3 of the bonding component 2 includes a first face 4 and an oppositely directed second face 5. The fixed portion 3 of the bonding component 2 further includes at opposite ends, a first distal region 14 and a second distal region 15. The fixed portion 3 and the fold-back portions 6A and 6B are a single continuous piece. Fastening components 12A and 12B are each connected to the distal region 7 of each of the bonding component 2 fold-back portions 6A and 6B respectively by an adhesive 8. Each fastening component 12A and 12B includes a first face 16 and an oppositely directed second face 17. The distal region 7 of each fold-back portion 6A and 6B of the bonding component 2 includes a first face 9 and an oppositely directed second face 10. Disposed on the first face 4 of the bonding component 2 is a release material 18. On a portion of each fastening component 12A and 12B is a fastening zone 11. The fastening zone 11 includes an adhesive 8 and a plurality of hooks 13. The fastening zone 11 is directed towards the first face 4 of the fixed portion 3 of the bonding component 2, and the fastening zone 11 is attached to the fixed portion 3 of the bonding component 2 by the adhesive 8 and hooks 13 of the fastening zone 11. Optionally, the fastening component 12 can be further attached to the bonding component 2 by an adhesive region 8 on the second face 17 of each fastening component 12A and 12B, located between each fastening zone 11 and the point of attachment of the fastening component 12 to the distal region 7 of each bonding component 2 fold-back portion 6A and 6B.The fastening zone 11 is attachable to a substrate (not shown), such as for example the surface of a hybrid diaper insert pad or the surface of a hybrid diaper shell, by the adhesive 8 and hooks 13 of the fastening zone 11. At least a portion of the second face 5 of the fixed portion 3 of the bonding component 2 is coated with an adhesive 8. Two of the portions of adhesive 8 form anti-flagging zones 25 on the bonding component 2. Two extension components 20A and 20B are each attached to a portion of the second face 5 of the fixed portion 3 of the bonding component 2 by an adhesive 8. Each extension component 20A and 20B includes a first face 23 and an oppositely directed second face 24, and an outwardly directed first distal region 26 and an inwardly directed second distal region 27. The second distal region 27 of each extension component 20A and 20B includes a fold-back portion 28. Disposed on the first face 23 of each extension component 20A and 20B is a release material 18. The anti-flagging zones 25 on the bonding components 2 are each directed towards the first face 23 of the respective extension component 20A and 20B, and each anti-flagging zone 25 is attached to the release material 18 on the respective extension component 20A and 20B by the adhesive 8 of the anti-flagging zone 25. Each anti-flagging zone 25 is attachable to a substrate (not shown), such as for example the surface of a hybrid diaper shell, by the adhesive 8 of the anti-flagging zone 25. An adhesive 8 on the second face 24 of each extension component 20A and 20B is attachable to a substrate (not shown), such as for example the surface of a hybrid diaper insert pad.

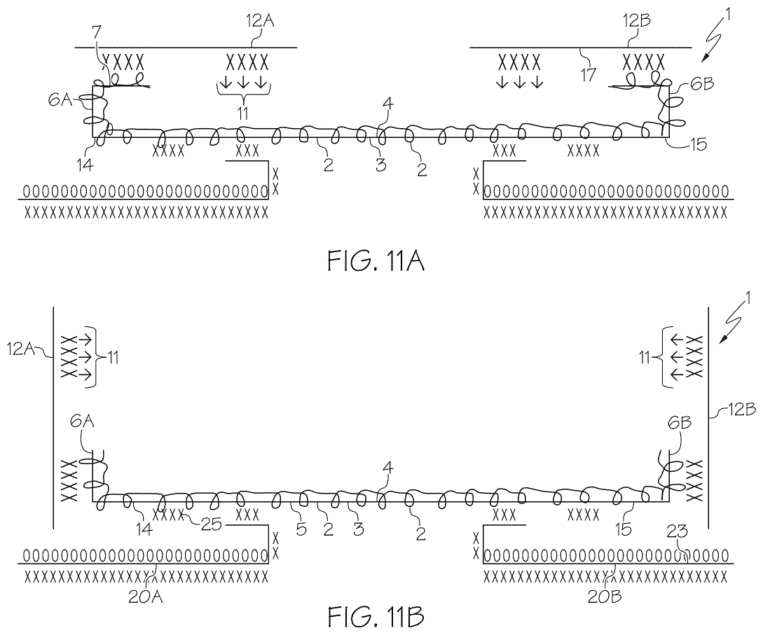

FIG. 10 exemplifies the embodiments of the fixation system 1 such as that described in FIG. 9, where rather than an adhesive and hook fastening zone, the fastening zone 11 on the fastening components 12A and 12B include an adhesive 8.

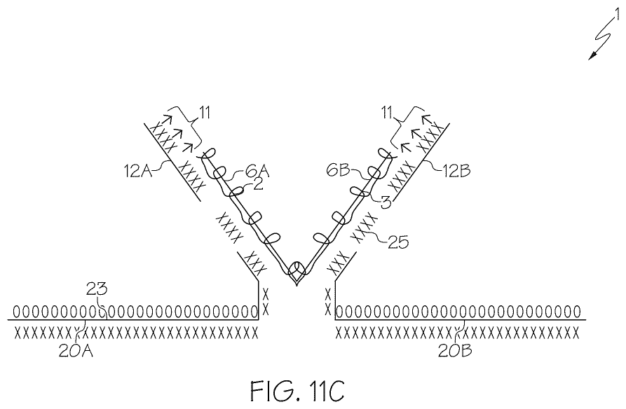

FIG. 11A exemplifies the embodiments of a fixation system 1 similar to that described in FIG. 9, where the bonding component 2 is made of a nonwoven (visually represented as a line encircling a straight line) that does not have disposed thereupon a release material, and the fixation system 1 is in a protection mode, with each fastening zone 11 protected between the second face 17 of its corresponding fastening component 12A and 12B, and the first face 4 of the fixed portion 3 of the bonding component 2. The distal region 7 of each fold-back portion 6A and 6B of the bonding component 2 lies parallel to the corresponding distal region 14 and 15 of the fixed portion 3 of the bonding component 2. FIG. 11B shows the fixation system 1 of FIG. 11A with the fastening zone 11 having been separated from the first face 4 of the fixed portion 3 of the bonding component 2. In this configuration each fold-back portion 6A and 6B of the bonding component 2 does not lie parallel to or substantially parallel to the distal regions 14 and 15 of the fixed portion 3 of the fixation system 1. Likewise, the fastening components 12A and 12B do not lie parallel to the fixed portion 3 of the bonding component 2. The anti-flagging zones 25 continue to attach the first face 23 of each extension component 20A and 20B to the second face 5 of the bonding component 2. The fastening zones 11 are exposed and available for attachment to a substrate, such as for example the surface of a hybrid diaper shell. FIG. 11C shows the fixation system 1 of FIGS. 11A and 11B, with the fixation system 1 in extension mode. The fold-back portion 6A and 6B of each bonding component 2 is fully extended. The anti-flagging zones 25 are separated from the first face 23 of the extension components 20A and 20B, and the fixation system 1 is fully extended so that the exposed fastening zones 11 on the fastening components 12A and 12B, and the exposed anti-flagging zones 25 can be attached to a substrate, such as for example the surface of a hybrid diaper pad, for disposal thereof. In this configuration the fixed portion 3 of the bonding component 2 does not lie parallel to or substantially parallel to at least a portion of the extension components 20A and 20B.

In some embodiments, the anti-flagging zone is disposed on at least a portion of the fold-back portion of the bonding component. In this configuration, the bonding component fold-back portion has a distal region that includes a secondary fold-back portion. The secondary fold-back portion is attached to the fastening component of the fixation system. The secondary fold-back portion can be attached to the fastening component by any suitable means. In some embodiments, the first face of the distal region of the secondary fold-back portion is attached to the second face of the fastening component. In some embodiments, the bonding component fold-back portion is at least as long as the fixed portion of the bonding component. In some embodiments, the bonding component fold-back portion is as long as the fixed portion of the bonding component. In some embodiments, the bonding component fold-back portion is longer than the fixed portion of the bonding component. In some embodiments, the bonding component fold-back portion is shorter than the fixed portion of the bonding component. In some embodiments, the bonding component fold-back portion and the fixed portion of the bonding component are a single component. In some embodiments, the bonding component fold-back portion and the fixed portion of the bonding component are separate components that are attached to each other by any suitable means. In some embodiments, when the fixation system is in a protection mode, the bonding component fold-back portion is oriented so that at least a portion of the bonding component fold-back portion extends over the fastening component. In this orientation, the anti-flagging zone is located on at least a portion of the second face of bonding component fold-back portion. Thus, the anti-flagging zone is directed towards the first face of the fastening component. In this manner, the anti-flagging zone is protected from prematurely adhering to a substrate. The area between the second face of the bonding component fold-back portion, and the first face of the fastening component, serves as an area of protection for the anti-flagging zone. In some embodiments, in the protection mode, the fastening component lies parallel to or substantially parallel to the fold-back portion of the bonding component. The adhesive in the anti-flagging zone attaches the second face of the fold-back portion of the bonding component to the first face of the fastening component, thereby protecting the anti-flagging zone while the fixation system is in the protection mode. In some embodiments, disposed on at least a portion of the first face of the fastening component is a release material. Any suitable release material can be used, such as, for example, a silicone-based or fluor-based release material. The anti-flagging zone adhesive lightly attaches to the release material while the fixation system is in the protection mode. When the fixation system is to be prepared for use, the fastening zone is exposed from its area of protection. This can be achieved by separating the fastening zone from the fixed portion of the bonding component. With the fastening zone now exposed, the fixation system is in the fastening mode, where the fastening zone can be attached to a substrate, such as for example, a hybrid diaper shell. The fastening zone can be attached to the substrate by at least one of the fastening zone hooks or adhesive. The anti-flagging zone remains protected during the fastening mode, as the fold-back portion of the bonding component remains attached to the fastening component. When the fixation system is to be prepared for disposal, the fastening zone is detached from the surface to which it had been attached, such as for example the shell of a hybrid diaper, and the anti-flagging zone is exposed from its area of protection. This can be achieved by separating the anti-flagging zone from the fastening component. With the anti-flagging zone now exposed, the fixation system is in the extension mode, where the anti-flagging zone can be attached to a substrate, such as for example, the outer surface of a hybrid diaper insert pad. Thus, even though there is no separate extension component, the extended fold-back portion of the bonding component, along with the extended fastening component, act as an extension component or extension system on the fixation system. Accordingly, in some embodiments, the extension system includes at least a portion of the fold-back portion of the bonding component, and the fastening component.

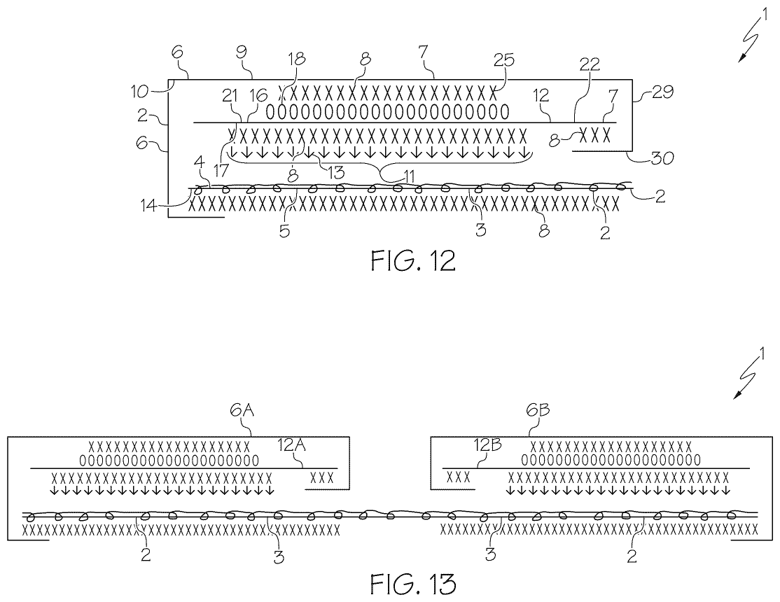

As exemplified in FIG. 12, in some embodiments, the fixation system 1 includes a bonding component 2 that includes a fixed portion 3 and a fold-back portion 6; and a fastening component 12. The bonding component 2 fixed portion 3 and the bonding component fold-back portion 6 are individual pieces. The fixed portion 3 of the bonding component 2 has a first face 4 and an oppositely directed second face 5. An adhesive 8 is disposed on the second face 5 of the fixed portion 3 of the bonding component 2. A portion of the adhesive 8 on the second face 5 attaches the first distal region 14 of the fixed portion 3 of the bonding component 2 to the fold-back portion 6. The fold-back portion 6 of the bonding component 2 includes a distal region 7. The distal region 7 of the fold-back portion 6 includes a first face 9 and an oppositely directed second face 10. The distal region 7 further includes a secondary fold-back portion 29 having a distal region 30. The fastening component 12 includes a first face 16 and an oppositely directed second face 17, a first distal region 21, and a second distal region 22. A portion of the distal region 30 of the secondary fold-back portion 29 is attached to the second face 17 of the fastening component 12 by an adhesive 8. Disposed on a portion of the second face 17 of the fastening component 12 is the fastening zone 11. The fastening zone 11 includes an adhesive 8 and a plurality of hooks 13. The bonding component 2 is made of a nonwoven material (visually represented as a line encircling a straight line). The fastening zone 11 is directed towards the first face 4 of the fixed portion 3 of the bonding component 2, and the fastening zone 11 is attached to the fixed portion 3 of the bonding component 2 by the adhesive 8 and hooks 13 of the fastening zone 11. The fastening zone 11 is attachable to a substrate (not shown), such as for example the surface of a hybrid diaper insert pad or the surface of a hybrid diaper shell, by the adhesive 8 and hooks 13 of the fastening zone 11. The adhesive 8 on the second face 5 of the fixed portion 3 of the bonding component 2 is attachable to a substrate (not shown), such as for example the surface of a hybrid diaper insert pad. Disposed on a portion of the first face 16 of the fastening component 12 is a release material 18. Disposed on a portion of the second face 10 of the fold-back portion 6 of the bonding component 2 is an anti-flagging zone 25. The anti-flagging zone 25 includes an adhesive 8. The anti-flagging zone 25 is directed towards the first face 16 of the fastening component 12, and the anti-flagging zone 25 is attached to the release material 18 on the first face 16 of the fastening component 12 by the adhesive 8 of the anti-flagging zone 25. The anti-flagging zone 25 is attachable to a substrate (not shown), such as for example the surface of a hybrid diaper shell.

As described hereinabove, in some embodiments, at least one portion of the fixation system mirrors at least one other portion of the fixation system. FIG. 13, exemplifies embodiments of the fixation system 1 described in FIG. 12, including a mirrored bonding component 2 and fastening components 12A and 12B. The mirrored fixed portions 3 of the bonding component 2 are a single component, while the fixation system 1 includes two independent fastening components 12A and 12B, and two independent fold-back portions 6A and 6B of the bonding component 2. Each side of the fixation system is identical to the other side, but reversed, as if reflected in a mirror.

As noted herein-above, any of the components, or parts of components, can be mirrored. In some embodiments, a single component has two mirrored sections. In some embodiments, two components mirror each other. In some embodiments, the fixation system includes at least one of two bonding components, two fastening components or two extension components. In some embodiments, at least one of the members of the two bonding, the two fastening components and the two extension components, mirror each other, or substantially mirror each other. However, it will be understood that there are embodiments where each side of the fixation system is not identical to the other, whether in dimension, arrangement of the components, or even presence of certain components. For example, in some embodiments, at least one component on one side of the fixation system can be shorter than the corresponding component on the other side of the fixation system. In some embodiments, at least one component on one side of the fixation system can be longer than the corresponding component on the other side of the fixation system. In some embodiments, one side of fixation system can include a secondary fold-back portion and an anti-flagging zone, while the other side does not.

As noted hereinabove, there are several locations on the fixation system on which an adhesive can be used. Any suitable adhesive can be used in any of these locations, such as for example those described hereinabove. In some embodiments, different types of adhesive are used in different locations on the fixation system. In some embodiments, different types of adhesive are used in at least two locations on the fixation system. In some embodiments, the same type of adhesive is used in each location on the fixation system. In some embodiments, the same type of adhesive is used in at least two locations on the fixation system.

As noted herein-above, a release material can be disposed on a face of any of the components of the fixation system. In some embodiments, a release material is disposed on at least one of the first face of the extension component, the first face of the fixed portion of the bonding component, or the first face of the bonding component.

Also disclosed is an article including a substrate upon which is attached the above described fixation system. Suitable substrates include, for hybrid diaper insert pads and hybrid diaper shells. The fixation system can be attached to a substrate by the fastening zone or the anti-flagging zone, in any suitable manner, many of which are described hereinabove. In some embodiments, the hybrid diaper insert pads include leg cuffs to provide added protected against soiling materials on the hybrid diaper.

Also disclosed is a hybrid diaper including a diaper shell and the hybrid diaper insert pad described above, where the fastening zone on the fixation system attaches the diaper shell to the fixation system.

Also disclosed is a method of using the fixation system of the disclosure. The method includes providing the fixation system. In some embodiments the second face of the bonding component fixed portion is attached to a first substrate, such as for example the surface of a hybrid diaper insert pad. In some embodiments the second face of the extension component is attached to a first substrate, such as for example the surface of a hybrid diaper insert pad. The fastening zone is separated from the first face of the fixed portion of the bonding component. In those embodiments where a release material is disposed on the first face of the fixed portion of the bonding component, the fastening zone is separated from the release material on the first face of the fixed portion of the bonding component. The separation of the fastening zone from the bonding component exposes the fastening zone from its area of protection. The exposed fastening zone is attachable to any suitable substrate. In some embodiments, the fastening zone is attached to a second substrate, such as for example the surface of a hybrid diaper shell. Thus, the fixation system can securely attach the hybrid diaper insert pad to the hybrid diaper shell.

In those embodiments where the fixation system includes an extension component, or a bonding component fold-back portion with a secondary fold-back portion attached to a fastening component, the fixation system will be long enough to use in preparation for disposal of the hybrid diaper insert pad, by attachment on the outer surface of the hybrid diaper insert pad. In some embodiments, after the hybrid diaper insert pad has been used, the fixation system is used to prepare the hybrid diaper insert pad for disposal. The fastening zone is detached from the second substrate, such as for example the surface of the hybrid diaper shell. The anti-flagging zone is exposed. In some embodiments, the anti-flagging zone is exposed by separating the fixed portion of the bonding component from the extension component. In some embodiments, the anti-flagging zone is exposed by separating the fastening component from the extension component. In some embodiments, the anti-flagging zone is exposed by separating the fold-back portion of the bonding component from the fastening component. At least a portion of the fixation system is extended around at least a portion of the article to which the fixation system is attached, such as for example a hybrid diaper insert pad. In some embodiments, the previously detached fastening zone is reattached to the surface of the first substrate, such as for example the surface of a hybrid diaper insert pad. Due to the orientation of the fastening zone, a portion of the fixation system will need to be twisted to orient to fastening zone so that it can attach to the outer surface of the hybrid diaper insert pad. In some embodiments, instead of the fastening zone, the anti-flagging zone is attached to the surface of the first substrate. Thus, either the fastening zone or the anti-flagging zone can be used to securely close a hybrid diaper insert pad that has been prepared for disposal, for example by rolling up the hybrid diaper insert pad.

The present subject matter includes all operable combinations of features and aspects described herein. Thus, for example if one feature is described in association with an embodiment and another feature is described in association with another embodiment, it will be understood that the present subject matter includes embodiments having a combination of these features.

As described hereinabove, the present subject matter solves many problems associated with previous strategies, systems and/or devices. However, it will be appreciated that various changes in the details, materials and arrangements of components, which have been herein described and illustrated in order to explain the nature of the present subject matter, may be made by those skilled in the art without departing from the principle and scope of the claimed subject matter, as expressed in the appended claims.

1. A fixation system comprising,
at least one bonding component comprising
at least one fixed portion comprising a first face and an oppositely directed second face,
at least one fold-back portion comprising a distal region; and
optionally, at least one fastening component connected to the distal region of the bonding component fold-back portion,
wherein disposed on at least a portion of the second face of the fixed portion is an adhesive,
wherein the distal region of the fold-back portion comprises a first face and an oppositely directed second face,
wherein the fastening component comprises a first face and an oppositely directed second face,
wherein disposed on at least one of, at least a portion of the distal region of the fold-back portion, and at least a portion of the fastening component, is at least one fastening zone, and
wherein the fastening zone is directed towards the first face of the fixed portion of the bonding component.

2. The fixation system according to claim 1, wherein the distal region of the bonding component fold-back portion comprises a secondary fold-back portion,
wherein the secondary fold-back portion comprises a distal region,
wherein the secondary fold-back portion distal region comprises a first face and an oppositely directed second face, and
wherein the fastening component is attached to the secondary fold-back portion distal region.

3. The fixation system according to claim 2, wherein the fastening component is attached to the first face of the secondary fold-back portion distal region.

4. The fixation system according to claim 1, wherein the fastening zone comprises at least one of an adhesive and a plurality of hooks.

5. The fixation system according to claim 1, wherein the fastening zone is removably attached to the first face of the fixed portion of the bonding component.

6. The fixation system according to claim 1, wherein the fastening zone is attachable to a substrate.

7. The fixation system according to claim 1, wherein the fastening zone is disposed on at least a portion of the second face of the distal region of the fold-back portion of the bonding component.

8. The fixation system according to claim 1, wherein the fastening zone is disposed on at least a portion of the second face of the fastening component.

9. The fixation system according to claim 1, wherein the at least one bonding component is a single bonding component having two fold-back portions.

10. The fixation system according to claim 1, wherein the fixed portion and the fold-back portion are a single continuous piece.

11. The fixation system according to claim 1, wherein the fixed portion and the fold-back portion are independent pieces connected to each other.

12. (canceled)

13. The fixation system according to claim 1, wherein disposed upon at least a portion of the first face of the fixed portion of the bonding component is at least one of a plurality of hooks and an adhesive.

14. (canceled)

15. The fixation system according to claim 1, further comprising an extension system comprising at least one extension component comprising a first face and an oppositely directed second face, wherein the extension component is attached to at least one of the bonding component and the fastening component.

16. The fixation system according to claim 15, wherein the fastening component comprises at least one fold-back portion, and wherein at least a portion of the fold-back portion of the fastening component is attached to the extension component.

17. The fixation system according to claim 1, wherein the bonding component comprises a fold-back portion, and wherein at least a portion of the bonding component fold-back portion is attached to the fastening component.

18. The fixation system according to claim 1, wherein disposed on at least a portion of at least one of the first face of the extension component, the first face of the fixed portion of the bonding component, and the first face of the fastening component, is a release material.

19. The fixation system according to claim 1, wherein disposed upon at least a portion of at least one of the second face of the fixed portion of bonding component, the second face of the extension component, and the second face of the fold-back portion of the bonding component, is an anti-flagging zone.

20. The fixation system according to claim 19, wherein the anti-flagging zone comprises at least one of an adhesive and a plurality of hooks.

21. The fixation system according to claim 19, wherein the anti-flagging zone comprises an adhesive.

22. The fixation system according to claim 1, wherein the fixation system comprises at least one of two bonding components, two fastening components and two extension components.

23. The fixation system according to claim 1, wherein at least one of the members of the two bonding, the two fastening components and the two extension components, mirror each other, or substantially mirror each other.

24. An article comprising a substrate upon which is attached the fixation system according to claim 1.

25. The article according to claim 24, wherein the fastening zone attaches the fixation system to the substrate.

26. The article according to claim 24, wherein the substrate is one of a surface of a hybrid diaper insert pad and a surface of a hybrid diaper shell.

27. (canceled)

28. A hybrid diaper comprising a diaper shell and the hybrid diaper insert pad according to claim 26, wherein the fastening zone attaches the diaper shell to the fixation system.

29. A method of using the fixation system according to claim 1, comprising the steps of:
a. providing the fixation system;
b. adhering the second face of the bonding component fixed portion or the second face of the extension component to a first substrate;
c. separating the fastening zone from the fixed portion of the bonding component, thereby exposing the fastening zone; and
d. attaching the fastening zone to a second substrate.

30. The method according to claim 29, wherein the first substrate is a hybrid diaper insert pad.

31. The method according to claim 29 wherein the second substrate is a hybrid diaper shell.

32. The method according to claim 29, further comprising the steps of:
a. detaching the fastening zone from the second substrate;
b. exposing the anti-flagging zone;
c. extending the fixation system around at least a portion of an article to which the fixation system is attached; and
d. attaching at least one of the fastening zone and the anti-flagging zone to the first substrate.
